(19) United States
(12) Patent Application Publication
(10) Pub. No.: US 2024/0326268 A1
(43) Pub. Date: Oct. 3, 2024

(54) DRAPE

(71) Applicant: RIVERFIELD INC., Tokyo (JP)

(72) Inventor: Naoya MORITA, Tokyo (JP)

(73) Assignee: RIVERFIELD INC., Tokyo (JP)

(21) Appl. No.: 18/737,492

(22) Filed: Jun. 7, 2024

Related U.S. Application Data
(63) Continuation of application No. PCT/JP2021/045105, filed on Dec. 8, 2021.

(51) Int. Cl. B25J 19/00

(52) U.S. Cl. CPC B25J 19/0083

(57) ABSTRACT

A drape for covering a medical robot includes an attachment detachably attached to the medical robot, a flexible sheet covering the medical robot, the flexible sheet being fixed to the attachment, and a flexible body that prevents the sheet from being pinched between the attachment and the medical robot.

### CROSS-REFERENCE TO RELATED APPLICATION

This Application is a continuation of International Application No. PCT/JP2021/045105, filed on Aug. 8, 2021 in the Japan Patent Office, the contents of which being incorporated by reference herein in its entirety

### BACKGROUND

The present disclosure relates to a drape that isolates a medical robot from a surgical instrument.

Surgery using medical robots enhances the possibility of not only reducing the burden on a surgeon but also the burden on a patient. The possibility of remote medical care through highly accurate and stable treatment is also enhanced. Surgical instruments that come into direct contact with patients are generally configured to be sterilized after being used so the surgical instruments can be re-used, but the medical robot provided with mechanisms to move surgical instruments is generally not sterilized because the electrical and electronic components on the robot have no sterilization resistance. In the surgery using a medical robot, therefore, the portion of the room with the medical robot is covered with a drape that includes a soft and flexible sheet. The interior (unclean area) covered with the drape and an area (clean area) in which the surgical instrument exposed from the drape are isolated from each other to maintain respective environments of the clean area and the unclean area.

### SUMMARY

It is an aspect to provide a drape that can avoid a pinch of the drape sheet in a state in which the drape is attached to the medical robot, thereby preventing poor fixation of the attachment and damage to the drape sheet.

According to an aspect of one or more embodiments, there is provided a drape for covering a medical robot, the drape comprising an attachment detachably attached to the medical robot; a flexible sheet covering the medical robot, the flexible sheet being fixed to the attachment; and a flexible body that prevents the sheet from being pinched between the attachment and the medical robot.

### BRIEF DESCRIPTION OF THE DRAWINGS

FIG. 1 is a perspective view of a medical robot according to some embodiments.

FIG. 2 is a perspective view of an insertion member according to some embodiments.

FIG. 3 is a plan view of a driving portion of the medical robot according to some embodiments.

FIG. 4 is a cross-sectional view of a connection configuration via a separator according to some embodiments.

FIG. 5 is an exploded perspective view of the arm tip side of the medical robot according to some embodiments.

FIG. 6 is an exploded perspective view of a bearing structure according to some embodiments.

FIGS. 7A and 7B are plan views of a drape according to some embodiments.

FIG. 8 is a perspective view of a pinch preventer according to some embodiments.

FIGS. 9A and 9B are cross-sectional views of the pinch preventer according to some embodiments.

FIG. 10 is a schematic cross-sectional view of a connection portion between a second sheet and a rotating member according to some embodiments.

FIG. 11 is a plan view of an opening of the second sheet and the pinch preventer according to some embodiments.

FIG. 12 is a perspective view of the second sheet and the separator before adhesion according to some embodiments.

FIG. 13 is a perspective view of the second sheet and the separator after adhesion according to some embodiments.

FIG. 14 is a schematic diagram of the coupling between the first sheet and the second sheet according to some embodiments.

FIG. 15 is a perspective view of an attachment portion of the first sheet according to some embodiments.

### DETAILED DESCRIPTION

As discussed above, a drape that isolates the clean area and the unclean area from each other may be provided with an attachment that transmits the operation of the drive mechanism of the medical robot to a member provided with the surgical instrument. When used, the attachment is attached to a predetermined position on the robot, but there is a concern that a flexible sheet continuous with the attachment may be pinched between the attachment and the robot during the attaching operation. If such a pinch occurs, it may cause poor fixation of the attachment or damage to the drape sheet.

It is an aspect to provide a drape that can avoid a pinch of the drape sheet in a state in which the drape is attached to the medical robot, thereby preventing poor fixation of the attachment and damage to the drape sheet. According to the various embodiments described therein, a drape can avoid poor fixation to the attachment and can avoid damage because a pinch of the drape sheet when the drape is attached to the medical robot is avoided.

According to some embodiments, a drape for covering a medical robot may include an attachment attached to a connection portion of the medical robot, a flexible sheet covering the medical robot, the sheet being inseparable from the attachment, and a joint and a separator each provided on the attachment and configured to prevent the sheet from being pinched between the attachment and the connection portion of the medical robot.

By providing such a joint and a separator, it is possible to prevent the flexible sheet from being pinched between the attachment and the connection portion when covering the medical robot with the drape.

According to some embodiments, the joint and the separator may each have a flexibility greater than a flexibility of the attachment and less than a flexibility of the sheet. By providing the joint and the separator with appropriate flexibility in this way, it is possible to prevent the sheet from coming too close to the attachment.

According to some embodiments, the joint and the separator may be translucent. This configuration allows the attachment situation between the attachment and the connection portion to be referred to via the joint and the separator.

According to some embodiments, the separator may include a plate having an extended portion extending outward from an outer surface of the attachment. The plate can prevent the sheet from being pinched in the area outside the outer surface of the attachment.

According to some embodiments, the plate may be inseparably attached to the sheet in an area including an end portion of the extended portion. This configuration allows the clean area and the unclean area to be reliably isolated between the plate and the sheet.

According to some embodiments, a first adhesive layer may be provided on at least a part of an outer surface of the separator, the sheet and the separator may be inseparably attached to each other by the first adhesive layer. Thus, the sheet and the separator are inseparably attached to each other by the first adhesive layer, and the clean area and the unclean area can thereby be reliably isolated between the sheet and the separator.

According to some embodiments, the separator may include a plate having an extended portion extending outward from an outer surface of the attachment, and the first adhesive layer may be provided on a surface of the plate and extends to an end portion of the extended portion of the plate. This configuration allows the sheet and the plate to be attached to each other by the first adhesive layer up to the end portion of the extended portion, and the clean area and the unclean area can be reliably isolated.

According to some embodiments, the drape may further include a second adhesive layer provided on a side of the sheet opposite to a side to which the first adhesive layer is attached, and the sheet may include an interposed portion interposing between the first adhesive layer and the second adhesive layer. Thus, the sheet is interposed between the first adhesive layer and the second adhesive layer by the interposed portion, and the clean area and the unclean area can thereby be reliably isolated.

According to some embodiments, the attachment may be on a side of the second adhesive layer opposite to a side facing the sheet. This configuration allows the attachment to adhere by the second adhesive layer, and the clean area and the unclean area can be reliably isolated between the attachment and the sheet.

According to some embodiments, a cover film may be provided on a side of the second adhesive layer opposite to a side facing the sheet and configured to protect the second adhesive layer. This configuration can prevent the second adhesive layer from adhering to an unintended site at a stage before the sheet is adhered to the attachment.

According to some embodiments, the interposed portion of the sheet may include a folded portion of the sheet. This configuration allows the sheet including its folded portion to be interposed between the first adhesive layer and the second adhesive layer, and the clean area and the unclean area can be reliably isolated.

According to some embodiments. the drape may be further configured to cover a holder at an end of an arm of the medical robot, the holder may be detachable form the end of the arm, rotatable around the arm, and configured to hold an insertion member to be inserted into a patient. The attachment may include a bearing. The bearing may include a ring-shaped fixation member attached to the arm in a state in which the holder is inserted into the fixation member, and a ring-shaped rotating member slidably and rotatably attached to the fixation member in a state in which the holder is inserted into the rotating member and rotatable together with the holder. The sheet may include a first sheet attached to the fixation member and may be configured to cover the arm, and a second sheet attached to the rotating member and rotatable together with the rotating member, and may be configured to cover the holder. Accordingly, the sheet having the first sheet that covers the arm of the medical robot and the rotatable second sheet that covers the holder, it is possible to prevent jamming of the sheet when the drape is placed over the medical robot.

According to some embodiments, the joint may be on an outer periphery of the fixation member. Accordingly, when the first sheet is placed over the holder provided at the end of the arm of the medical robot, the joint can prevent jamming of the sheet on the outer periphery of the fixation member.

According to some embodiments, the separator may include a plate extending in a direction away from the rotating member. The plate may prevent jamming of the sheet in the area around the rotating member.

According to some embodiments, the rotating member may include a recessed portion on its outer periphery in which an adhesive layer is disposed. The adhesive layer may be configured to inseparably attach the rotating member and the second sheet to each other. By disposing the adhesive layer in such a recessed portion, it is possible to reliably isolate the clean area and the unclean area from each other at the adhesion portion between the rotating member and the second sheet.

According to some embodiments, the attachment may include a separator between the medical robot and an insertion member, the insertion member being held by the medical robot and configured to be inserted into a patient, the separator being configured to isolate the insertion member and the medical robot from each other and transmit power in forward and backward directions from a power transmitter of the medical robot to a movable part of the insertion member. The Separator may include a slider having a first engagement portion engaged with the power transmitter and a second engagement portion engaged with the movable part of the insertion member, the slider may be configured to transmit the power received from the power transmitter to the movable part, and a separator main body including a through-hole through which a part of the slider is inserted, the separator main body being detachably attached to the medical robot. Thus, in the configuration having the separator as the attachment, when covering the medical robot with the drape, the separator can prevent the flexible sheet from being pinched between the attachment and the connection portion.

According to some embodiments, the drape may include a first adhesive layer provided on a side of the separator facing the separator main body and a second adhesive layer provided on a side of the separator main body facing the separator. The sheet may include an interposed portion interposed between the first adhesive layer and the second adhesive layer. Thus, the sheet is interposed between the first adhesive layer and the second adhesive layer by the interposed portion, and the clean area and the unclean area can thereby be reliably isolated.

According to some embodiments, the separator may include an opening portion, and the sheet may be attached to the first adhesive layer so as not to cover the opening portion. With this configuration, the sheet is attached so as not to cover the through-hole of the separator.

According to some embodiments, the separator may include a plate. This configuration allows the plate to prevent the pinch of the sheet.

According to some embodiments, a portion of the plate located around the slider may extend to a length corresponding to an edge the separator main body. With this configuration, the effect of preventing the sheet from being pinched can be obtained without extending the plate-shaped body more than necessary.

Hereinafter, various embodiments will be described with reference to the drawings. In the following description, the same members are denoted by the same reference numerals and the description of members once explained may be omitted. In addition, each figure illustrates X-Y-Z coordinates as reference coordinates. In the following description, Z1-Z2 direction will be referred to as the up-down direction, X1-X2 direction will be referred to as the front-back direction, and Y1-Y2 direction will be referred to as the left-right direction. The X1-X2 direction and the Y1-Y2 direction are orthogonal to each other, and the X-Y plane including them is orthogonal to the Z1-Z2 direction. The state viewed from the upper side (Z1 side) to the lower side (Z2 side) may be referred to as a plan view.

### Configuration of Medical Robot

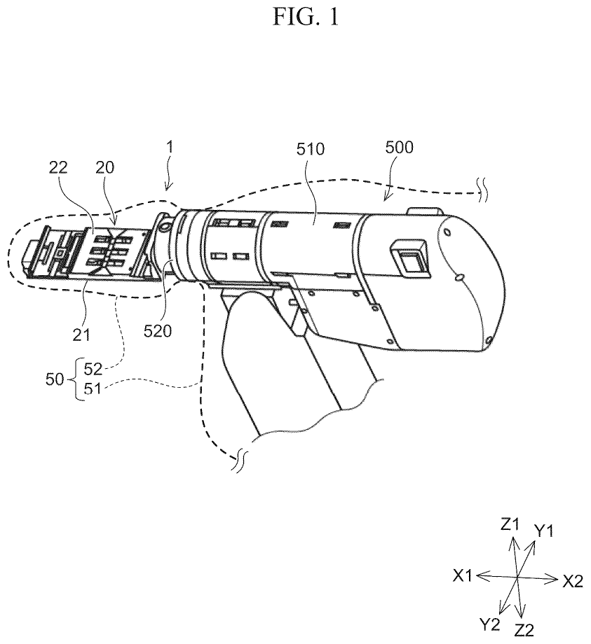

FIG. 1 is a perspective view of a medical robot according to some embodiments.

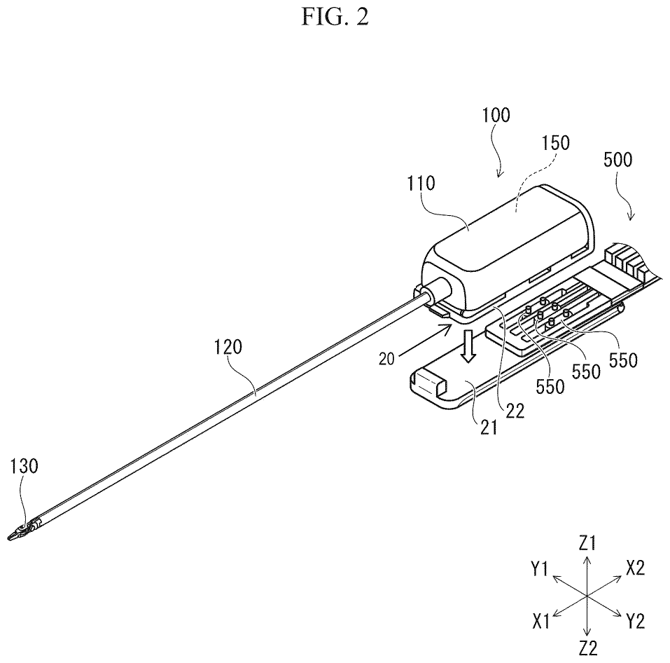

FIG. 2 is a perspective view of an insertion member according to some embodiments.

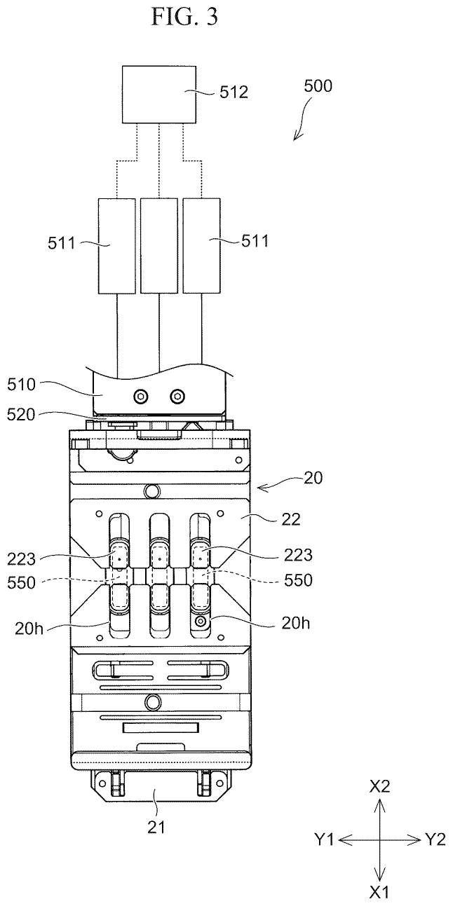

FIG. 3 is a plan view of a driving portion of the medical robot according to some embodiments.

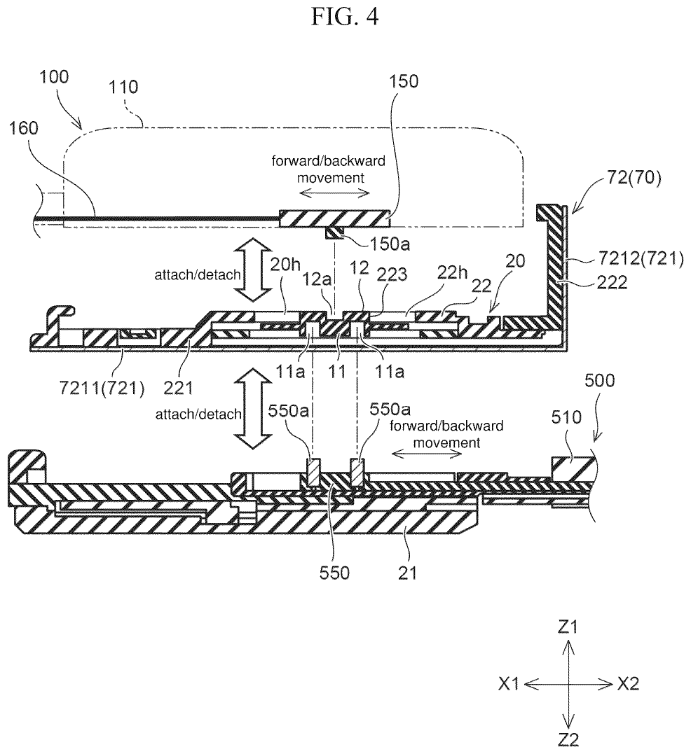

FIG. 4 is a cross-sectional view of a connection configuration via a separator according to some embodiments.

As illustrated in FIG. 1, a medical robot 500 may include an arm 510 that is a remotely-operable manipulator whose direction and angle can be changed. The tip of the arm 510 may be provided with a holder 21. In an embodiment, an attachment 20 may be attached to the holder 21. The attachment 20 may include a separator 22. However, this is only an example and, in some embodiments, the attachment 20 may include components in addition to the separator 22. From the front end of the arm 510, the holder 21 and the separator 22 of the attachment 20 extend forward. The attachment 20 may be arranged to cover the holder 21. The insertion member 100 (see FIG. 2) may be detachably attached to the attachment 20 of the arm 510. In an embodiment, the insertion member 100 may be attached to the holder 21 by the separator 22. The insertion member 100 may be inserted into the body of a patient who is an object of treatment with the medical robot 500. The posture of the insertion member 100 may be changed in accordance with changes in the direction and angle of the arm 510. The insertion member 100 may be used as a treatment instrument for the treatment and an endoscope.

As illustrated in FIG. 2, the insertion member 100 may include a main body 110, a shaft 120 extending from the main body 110, and a treatment part 130 provided at the tip of the shaft 120 (an end portion opposite to the main body 110). In some embodiments, the treatment part 130 may be a forceps. FIG. 2 illustrates a state in which the separator 22 of the attachment 20 is attached to the insertion member 100. At the time of actual treatment, the separator 22 may be first attached to the holder 21 of the medical robot 500 as illustrated in FIG. 1, and the insertion member 100 may then be attached to the separator 22 which has been attached to the holder 21.

The insertion member 100 attached to the medical robot 500 may be subjected to a sterilization treatment and placed in a clean area on the treatment room side. On the other hand, the medical robot 500 may be placed in an unclean area that is not as clean as the insertion member 100. For this reason, during the treatment, the medical robot 500 is covered with a sheet 50 of a drape 1 (see the broken line of FIG. 1) to isolate the clean area and the unclean area. The drape 1 is also subjected to a sterilization treatment.

Here, the drape 1 has the attachment 20 with the separator 22, and the sheet 50 being flexible. The sheet 50 may be inseparable from the attachment 20. Details of the drape 1 will be described later.

As illustrated in FIG. 3, the medical robot 500 may be provided with actuator 511 and a controller 512. The actuator 511 may generate driving force for operating the insertion member 100. The actuator 511 may be connected to respective power transmitters 550 that transmit driving force in the front-back direction (X1-X2 direction) to the attachment 20.

Various embodiments will be described as being applied to a state in which the actuator 511 generates driving force using a gas such as air or a fluid. The actuator 511 may use electric motors, and the type of generating the power is not limited. The actuator 511 may have a configuration using a piston and a cylinder or a configuration in which the driving force is generated from other known fluids, and the specific configuration is not limited.

The controller 512 controls generation of the driving force in the actuator 511. The controller 512 also controls the movement in the X direction in the power transmitters 550 (FIG. 2) and the arrangement positions of the power transmitters 550. Various embodiments will be described as being applied to a state in which the controller 512 controls the supply of a gas such as air to the actuator 511.

To attach the insertion member 100 to the holder 21, the main body 110 of the insertion member 100 may be fitted to the separator 22 attached to the holder 21 by being slid in the Y direction. However, this is only an example, and the attachment of the insertion member 100 to the holder 21 is not limited thereto.

By fitting the main body 110 to the separator 22, the power from the power transmitters 550 can be transmitted to the main body 110 via sliders 223 of the separator 22. Movable parts 150 are provided in the main body 110. The movable parts 150 are coupled with the treatment part 130 via wires 160 and can perform respective predetermined movements (e.g., movements in the front-back direction). Movements of the power transmitters 550 (e.g., movements in the front-back direction) are transmitted from the sliders 223 of the separator 22 to the movable parts 150 of the insertion member 100 and can operate the treatment part 130 via the wires 160 (e.g., can open and close the forceps). That is, the separator 22 may transmit the power in the forward and backward directions from the power transmitters 550 of the medical robot 500 to the movable parts 150 provided in the insertion member 100.

As illustrated in FIG. 4, the separator 22 has a separator main body 221 and sliders 223. The separator main body 221 is provided with through-holes 20h through which respective parts of the sliders 223 are inserted, and is detachably attached with respect to the medical robot 500. The sliders 223 are provided so as to be slidable in the front-rear direction (X1-X2 direction) with respect to the separator main body 221. The sliders 223 have first engagement portions 11 that engage with the power transmitters 550 and second engagement portions 12 that engage with the movable parts 150 of the insertion member 100, and transmit the power received from the power transmitters 550 to the movable parts 150.

The first engagement portions 11 of the separator 22 are provided with recessed portions 11a into which protruding portions (power-side protruding portions 550a) of the power transmitters 550 are fitted. When the separator 22 is attached to the holder 21 of the arm 510, the power-side protruding portions 550a of the power transmitters 550 are fitted into the recessed portions 11a of the first engagement portions 11 of the sliders 223. This configuration allows the power when the power transmitters 550 move forward/backward to be transmitted to the first engagement portions 11, and the sliders 10 can achieve the front-back movements.

The second engagement portions 12 are provided with recessed portions 12a into which protruding portions (insertion-side protruding portions 150a) of the movable parts 150 of the insertion member 100 are fitted. When the main body 110 of the insertion member 100 is attached to the separator 22, the insertion-side protruding portions 150a protruding from the back surface of the main body 110 are fitted into the recessed portions 12a of the second engagement portions 12. This configuration allows the sliders 223 and the movable parts 150 of the insertion member 100 to be engaged with each other, and the front-back movements of the power transmitters 550 can be transmitted from the sliders 223 to the movable parts 150 of the insertion member 100. That is, when the power transmitters 550 are moved in the front-back direction, the power is transmitted from the sliders 223 to the movable parts 150, and the movements of the movable parts 150 in the front-back direction can be transmitted to the wires 160 to operate the treatment part 130 via the wires 160.

### Bearing Structure of Drape

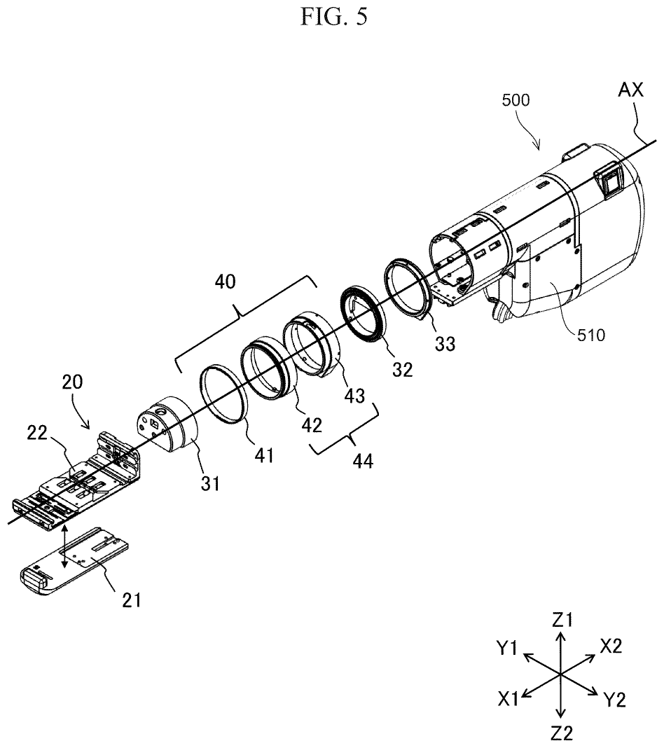

FIG. 5 is an exploded perspective view of the arm tip side of the medical robot according to some embodiments.

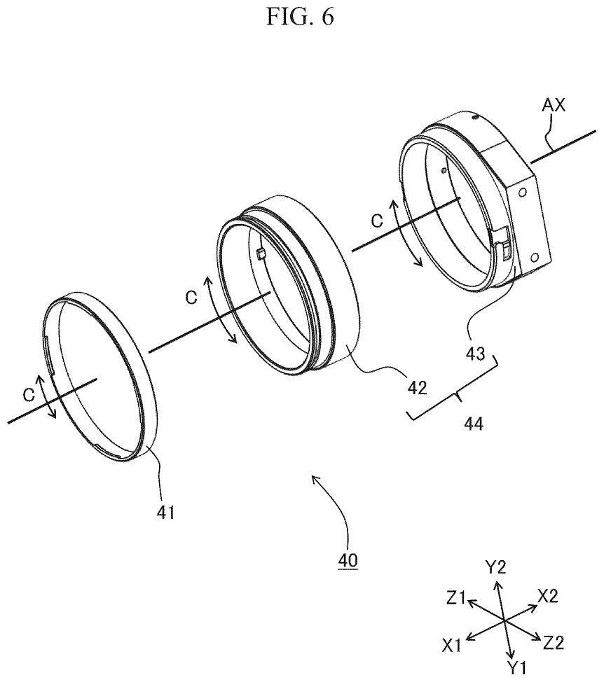

FIG. 6 is an exploded perspective view of a bearing structure according to some embodiments.

As illustrated in FIG. 5, the holder 21 and the separator 22 are attached to the arm 510 via a switch unit 31, a bearing structure 40, and a bearing member 32 that is arranged in this order from the front to the rear. The bearing member 32 may be fixed to the arm 510 via an annular bearing plate 33. In an embodiment, the holder 21 may be provided integrally with the switch unit 31. The separator 22 may be detachably attached to the holder 21. The bearing member 32 may be a rolling bearing whose outer ring is fixed to the arm 510 and whose inner ring can rotate relative to the outer ring.

As illustrated in FIGS. 5 and 6, the bearing structure 40 includes a rotating member 41, a joint member 42, and a base member 43 that are all ring-shaped. In some embodiments, the joint member 42 serves as a fixation member. The joint member 42 may be referenced as a joint. Rear portions of the holder 21 and the separator 22 may be inserted inside the rotating member 41, the joint member 42, and the base member 43 of the bearing structure 40. In some embodiments, the holder 21 and the separator 22 may be provided integrally with the rotating member 41.

The joint member 42 and the base member 43 may be detachably attached to each other by fitting and in some embodiments, the joint member 42 and the base member 43 may constitute a fixation member 44. The base member 43 may be attached with respect to the arm 510. The rotating member 41 may be attached with respect to the joint member 42 of the fixation member 44 by being slid along a circumferential direction C.

The base member 43 may comprise a metal from the viewpoint of durability against detachable attachment with the joint member 42, and in some embodiments, the metal may be aluminum or duralumin.

The joint member 42 and the rotating member 41 may comprise a resin material from the viewpoint of slidability and durability against sterilization, and in some embodiments, the resin material may be ABS resin, polycarbonate, and/or polyacetal.

By configuring the rotating member 41 and the fixation member 44 with the above-described materials, it is possible to ensure the slidability for a long period of time, and damage due to sliding over a long period of time can be reduced.

The holder 21, the separator 22, the switch unit 31, and the rotating member 41 may be supported by the bearing member 32 in a rotatable manner and are driven by a motor to rotate around an axis of rotation AX along the front-rear direction (X1-X2 direction).

In the drape 1, the detachable attachment of the sheet 50 may be performed via the attachment 20. The base member 43 may be fixed to the arm 510 side, and the rotating member 41 and the separator 22 on the tip side of the joint member 42 can be detachably attached with respect to the base member 43. According to some embodiments, the attachment 20 may include the separator 22, the rotating member 41, and the joint member 42.

### Configuration of Drape

Details of the drape 1 will be described herein.

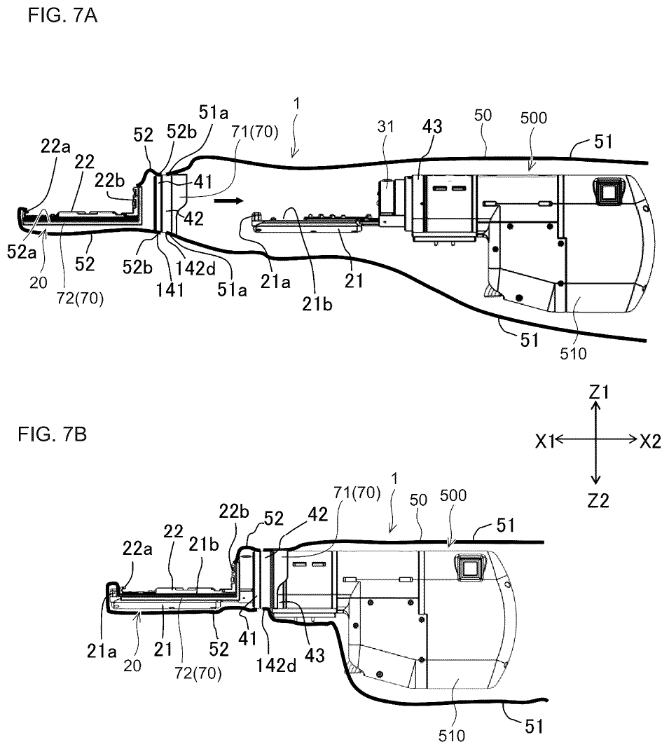

FIGS. 7A and 7B are plan views of the drape according to some embodiments.

FIG. 7A is a side view illustrating a state before the drape 1 is attached to the holder 21, and FIG. 7B is a side view illustrating a state in which the drape 1 is attached to the holder 21 side and the arm 510 side.

As illustrated in FIGS. 7A and 7B, during the treatment, the medical robot 500 may be covered with the drape 1 having an approximately tubular shape with a plurality of opening portions. Even when the medical robot 500 is covered with the drape 1, the insertion member 100 can be detachably attached to the holder 21. The holder 21 to which the insertion member 100 is attached can also be rotated.

The drape 1 according to some embodiments may include the attachment 20, the sheet 50, and a pinch preventer 70. The attachment 20 includes the separator 22 described previously. In some embodiment, the attachment 20 may include components in addition to the separator 22. The separator 22 is disposed between the medical robot 500 and the insertion member 100 to isolate the insertion member 100 and the medical robot 500 from each other, and serves also to transmit the power in the front-back direction from the power transmitters 550 of the medical robot 500 to the movable parts 150 (see FIG. 2) provided in the insertion member 100.

The sheet 50 may include a first sheet 51 fixed to the joint member 42 of the bearing structure 40 and a second sheet 52 fixed along the rotating member 41 of the bearing structure 40.

Specifically, a first opening portion 51a that is a front opening portion (on the X1 side in the X1-X2 direction) of the tubular first sheet 51 may be fixed along an outer peripheral surface 142d on the rear side of the joint member 42. The first sheet 51 extends rearward from the joint member 42 and covers the surgical robot 500 including the arm 510.

Here, the fixation member 44 (see FIG. 5) may comprise two components of the base member 43 fixed to the arm 510 and the joint member 42 that can be detachably attached to the base member 43, and it is therefore easy to detachably attach the first sheet 51 with respect to the arm 510.

A front opening portion 52a of the tubular second sheet 52 may be provided in a linked manner with the separator 22 so as to be fixed from its front end portion 22a to its rear end portion 22b. This configuration allows the separator 22 and the second sheet 52 to be integrated, and the lower surface of the separator 22 is placed in the second sheet 52.

On the other hand, a rear opening portion 52b of the second sheet 52 may be fixed along an outer peripheral surface 141 of the rotating member 41.

Thus, the separator 22 and the rotating member 41 are attached with respect to the arm 510 from the state in which the second sheet 52 is attached. In this operation, the holder 21 and the switch unit 31 may be inserted into the rotating member 41, and the separator 22 may be attached to an upper surface 21b of the holder 21. According to this step, as illustrated in FIG. 7B, the front surface and lower surface of the holder 21 may be covered with the second sheet 52 from a front end portion 21a located on the front side of the front end portion 22a of the separator 22, and the upper surface 21b is covered with the separator 22. When the rotating member 41 rotates relative to the fixation member 44, the second sheet 52 rotates integrally with the rotating member 41, the separator 22, and the holder 21.

### Pinch Preventer

The pinch preventer 70 may be provided inseparably from the attachment 20

and may be flexible. As described above, when the medical robot 500 is covered with the drape 1, the sheet 50 is likely to be pinched between the attachment 20 and the connection portion (e.g., the holder 21 and/or the base member 43). The pinch preventer 70 according to some embodiments may be a flexible body that effectively prevents the sheet 50 being pinched between the attachment 20 and the connection portion when covering the medical robot 500 with the drape 1.

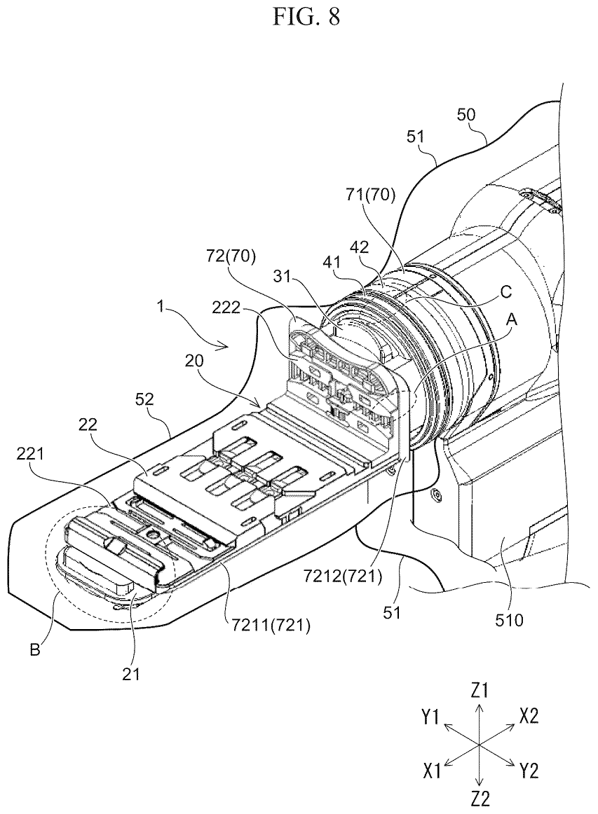

FIG. 8 is a perspective view of the pinch preventer according to some embodiments.

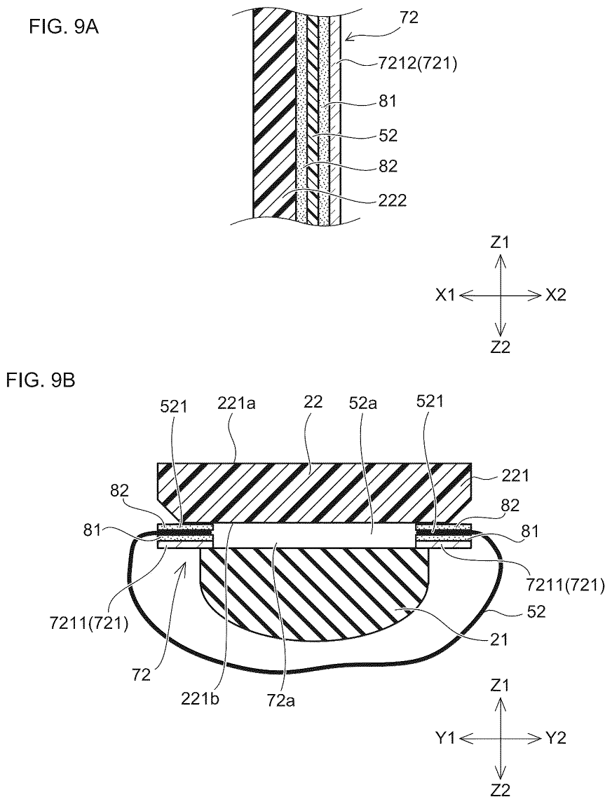

FIGS. 9A and 9B are schematic cross-sectional views of the pinch preventer. FIG. 9A illustrates a cross-sectional view of section A of FIG. 8 viewed in the Y1 direction, and FIG. 9B illustrates a cross-sectional view of section B of FIG. 8 viewed in the X2 direction.

The pinch preventer 70 may be a flexible body that is provided at a position corresponding to the connection portion between the arm 510 and the drape 1. According to some embodiments, the joint member 42 to which the first sheet 51 is connected may be provided with a first pinch prevention part 71. In addition, in some embodiments, the separator 22 to which the second sheet 52 is connected is provided with a second pinch prevention part 72. The first and second pinch prevention parts 71 and 72 are described in more detail below.

In a state in which the first sheet 51 is placed over the arm 510, the joint member 42, to which the first sheet 51 is connected, may be fitted to the base member 43. In this operation, the first pinch prevention part 71 may prevent the first sheet 51 from being pinched between the joint member 42 and the base member 43. In addition, in a state in which the second sheet 52 is placed over the holder 21 and the separator 22 is attached to the holder 21, the second pinch prevention part 72 may prevent the second sheet 52 from being pinched between the separator 22 and the holder 21 and may also prevent the second sheet 52 from being pinched between the separator 22 and the switch unit 31.

While it is desired to provide both the first pinch prevention part 71 and the second pinch prevention part 72, only one of them may be provided.

### Second Pinch Prevention Part

The second pinch prevention part 72 may include a flexible body. For example, in some embodiments, the second pinch prevention part 72 may include a plate-shaped body 721 having an extended portion that extends outward from the outer surface of the separator 22. The separator 22 may include a separator main body 221 that comes into contact with the holder 21, and an upright portion 222 which stands up from the separator main body 221 on a rear end side of the separator main body 221. In some embodiments, the second pinch prevention part 72 may include a first plate-shaped body 7211 that extends outward from the separator main body 221, and a second plate-shaped body 7212 that extends outward from the upright portion 222.

In some embodiments, the material of the plate-shaped body 721 may be polyethylene terephthalate (PET). A part of the plate-shaped body 721 having a flat plate shape may be folded thereby to configure the first plate-shaped body 7211 facing the separator main body 221 and the second plate-shaped body 7212 facing the upright portion 222. The flexibility of the second pinch prevention part 72 may be higher than that of the attachment 20 and lower than that of the sheet 50 (second sheet 52). Thus, the first plate-shaped body 7211 extends outward from the edge of the separator main body 221, and the second plate-shaped body 7212 extends outward from the edge of the upright portion 222, so that extended portions of the first plate-shaped body 7211 and second plate-shaped body 7212 serve as wing-shaped supports to prevent the second sheet 52 from approaching the separator 22.

Therefore, when the separator 22 is attached to the holder 21 in a state in which the sheet 50 is placed over the medical robot 500, the soft second sheet 52 can be prevented from approaching the edge of the separator 22 and can also be prevented from being pinched between the separator 22 and the holder 21 or between the separator 22 and the switch unit 31.

That is, when the separator 22 is attached to the holder 21, the first plate-shaped body 7211 prevents the second sheet 52 from approaching the edge of the separator main body 221 and also prevents the second sheet 52 from being pinched between the separator main body 221 and the holder 21.

When the separator 22 is coupled with the switch unit 31, the second plate-shaped body 7212 may prevent the second sheet 52 from approaching the edge of the upright portion 222 and also may prevent the second sheet 52 from being pinched between the upright portion 222 and the switch unit 31. Furthermore, in the state in which the separator 22 is coupled with the switch unit 31, the second plate-shaped body 7212 may extend in the direction away from the rotating member 41, so when the holder 21 and the attachment 20 rotate, it is possible to prevent the jamming of the second sheet 52 in an area around the rotating member 41.

In an embodiment, at least the extended portion of the plate-shaped body 721 of the second pinch prevention portion 72 may be translucent. Accordingly, when the separator 22 is attached to the holder 21, it is easy to refer to the attachment situation through the second pinch prevention part 72 having translucency.

As illustrated in FIGS. 9A and 9B, the plate-shaped body 721 is inseparably attached to the second sheet 52 in an area including the end portion of the extended portion. In some embodiments, the plate-shaped body 721 may be attached to the second sheet 52 via a first adhesive layer 81. The first adhesive layer 81 may be a double-sided tape. The first adhesive layer 81 may be provided on at least a part of the outer surface of the plate-shaped body 721 of the second pinch prevention portion 72. Thus, the second sheet 52 and the second pinch prevention part 72 are inseparably attached to each other by the first adhesive layer 81, and it is thereby possible to reliably isolate the clean area and the unclean area between the second sheet 52 and the second pinch prevention part 72.

In an embodiment, the first adhesive layer 81 may be provided on one main surface of the plate-shaped body 721 and may extend to the inner end portion of the extended portion of the plate-shaped body 721. This configuration allows the second sheet 52 and the plate-shaped body 721 to be attached to each other by the first adhesive layer 81 up to the end portion of the extended portion, and the clean area and the unclean area can be separated more reliably.

In some embodiments, a second adhesive layer 82 may be provided so as to cover at least a part of the portion of the second sheet 52 that is attached to the first adhesive layer 81. That is, the second adhesive layer 82 may be provided on the side of the second sheet 52 opposite to the side on which the first adhesive layer 81 is provided. The second adhesive layer 82 may be a double-sided tape. This configuration allows the second sheet 52 to have an interposed portion 521 (see FIG. 9B) interposed between the first adhesive layer 81 and the second adhesive layer 82. The second sheet 52 is attached to the separator 22 of the attachment 20 by the second adhesive layer 82 at the interposed portion 521.

In other words, the separator 22 of the attachment 20 may be adhered to the second adhesive layer 82 on the side opposite to the side facing the second sheet 52. This configuration allows the second sheet 52 to be interposed between the first adhesive layer 81 and the second adhesive layer 82 by the interposed portion 521, so that the clean area and the unclean area can be reliably isolated.

This interposed portion 521 may include a folded portion of the second sheet 52. The second sheet 52 may have the folded portion extending from the separator main body 221 to the upright portion 222 of the separator 22. In an embodiment, the interposed portion 521 may be provided at a folded portion of the second sheet 52 that faces the upright portion 222. This configuration allows the second sheet 52 including the folded portion of the second sheet 52 to be interposed between the first adhesive layer 81 and the second adhesive layer 82, and the clean area and the unclean area are reliably isolated.

As illustrated in FIG. 9B, in an embodiment, the portion of the plate-shaped body 721 located around the sliders 223 of the separator 22 (see FIG. 4) does not have a portion that protrudes further in a direction in the main surface of the plate-shaped body 721 than the separator main body 221 when the portion is viewed from the normal direction (Z1-Z2 direction, Z2 direction). In other words, an edge of a portion of the plate-shaped body 721 located around the sliders 223 may be coplanar with an edge the separator main body 221.

That is, a lower surface 221b of the separator main body 221 (the surface that contacts the holder 21) may be provided to be narrower than an upper surface 221a of the separator main body 221 (the surface that is opposite to the surface that contacts the holder 21). The plate-shaped body 721 may extend outward beyond the edge of the lower surface 221b of the separator main body 221, but not outward beyond the edge of the upper surface 221a of the separator main body 221. Therefore, when the separator 22 is viewed in the Z2 direction of the Z1-Z2 direction, the plate-shaped body 721 is hidden behind the separator main body 221 and cannot be seen. On the other hand, when the separator 22 is viewed in the Z1 direction of the Z1-Z2 direction, the plate-shaped body 721 is in a state of extending outward from the lower surface 221b of the separator main body 221. It is thereby possible to obtain the effect of preventing the second sheet 52 from being pinched without extending the plate-shaped body 721 more than necessary.

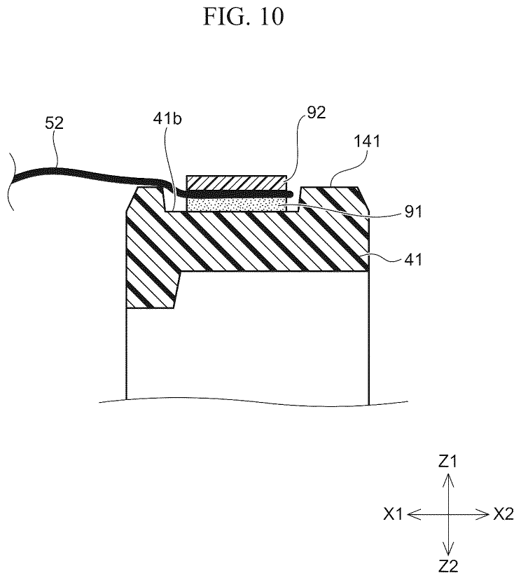

FIG. 10 is a schematic cross-sectional view of a portion between the second sheet and the rotating member. FIG. 10 illustrates a cross-sectional view of section C of FIG. 8 viewed in the Y1 direction.

As illustrated in FIG. 10, the end portion of the second sheet 52 may be connected to a recessed portion 41b provided in the outer peripheral surface 141 of the rotating member 41 of the bearing structure 40 with a double-sided tape 91. The recessed portion 41b may be provided along the entire circumferential direction of the rotating member 41. Therefore, the end portion of the second sheet 52 may be connected to the entire circumferential direction of the recessed portion 41b of the rotating member 41 with the double-sided tape 91. In some embodiments, the end portion of the second sheet 52 may be fixed from above with a tape 92 for pressing. This configuration allows the second sheet 52 and the rotating member 41 to be connected without a gap, and the clean area and the unclean area can be reliably isolated.

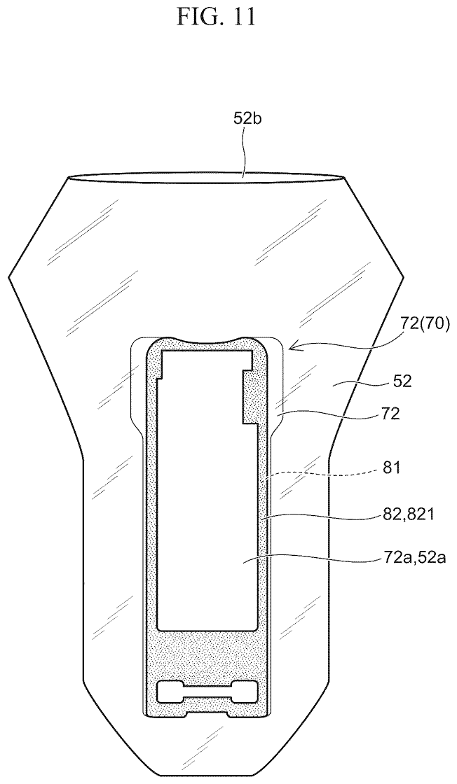

FIG. 11 is a plan view of an opening of the second sheet and the pinch preventer according to some embodiments.

FIG. 11 illustrates a state before the separator 22 is attached to the second sheet 52.

The front opening portion 52a may be provided in the central portion on the front surface side of the bag-shaped second sheet 52, and the separator 22 may be attached to cover the front opening portion 52a. The second pinch prevention part 72 may have the plate-shaped body 721 having a shape that extends outward from the edge of the front opening portion 52a. The second pinch prevention part 72 may be attached to the inside of the bag of the second sheet 52. That is, the second pinch prevention part 72 has an opening portion 72a and is attached to the inside of the bag of the second sheet 52 by the first adhesive layer 81 so that the opening portion 72a and the front opening portion 52a are aligned. This configuration allows the second sheet 52 and the second pinch prevention part 72 to be connected so that the second sheet 52 does not cover the opening portion 72a of the second pinch prevention part 72. In some embodiments, the second adhesive layer 82 may be attached to the outside of the bag of the second sheet 52 so as to rim the front opening portion 52a of the second sheet 52. The second adhesive layer 82 allows the separator 22 to be attached over the front opening portion 52a of the second sheet 52.

In an embodiment, in a state before the separator 22 is attached by the second adhesive layer 82, a cover film 821 that protects the second adhesive layer 82 may be provided on the side of the second adhesive layer 82 opposite to the side facing the second sheet 52. This configuration can prevent the second adhesive layer 82 from attaching to an unintended site before the separator 22 and the second sheet 52 are adhered together.

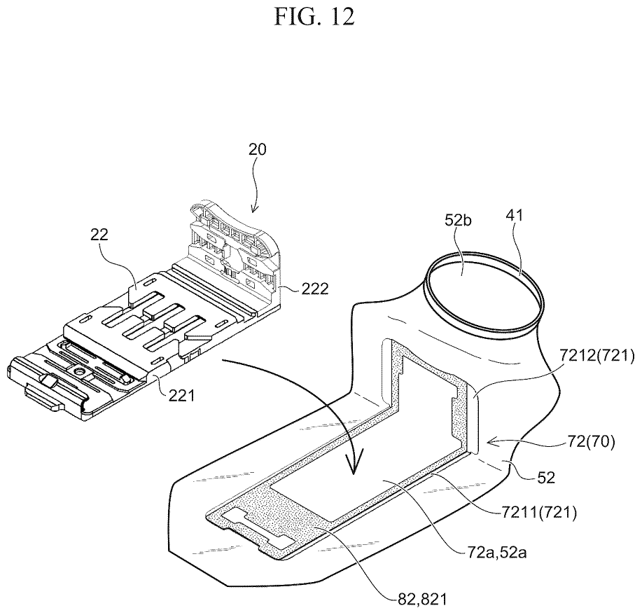

FIG. 12 is a perspective view of the second sheet and the separator before adhesion. To adhere the separator 22 to the second sheet 52, the second pinch prevention part 72 may be attached to the inside of the bag of the second sheet 52, and in this state a part of the rear end side of the second pinch prevention part 72 is folded approximately vertically. The second pinch prevention part 72 may be formed of PET, so the folded shape of the second pinch prevention part 72 is maintained due to the fold habit of PET. The folded portion faces the upright portion 222 of the separator 22.

In this state, the cover film 821 of the second adhesive layer 82 may be removed, and the separator 22 may be attached to the second sheet 52 using the second adhesive layer 82. This configuration allows the separator 22 to be attached to the position of the front opening portion 52a of the second sheet 52.

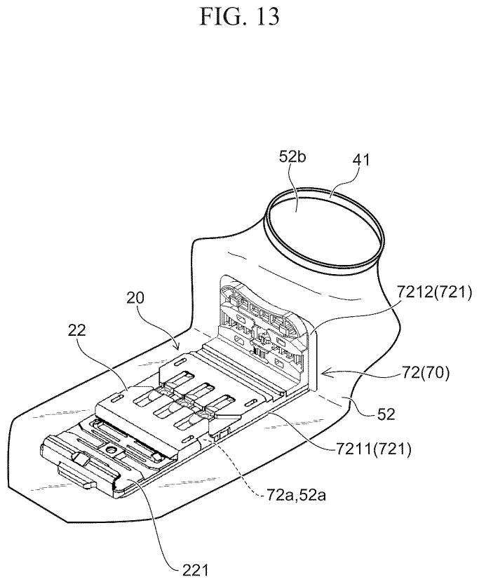

FIG. 13 is a perspective view of the second sheet and the separator after adhesion according to some embodiments.

In a state in which the separator 22 is attached to the second sheet 52, the second sheet 52 may be supported by the second pinch prevention part 72, and therefore is less likely to get pinched on the back side of the separator 22.

The ring-shaped rotating member 41 may be attached to the rear opening portion 52b of the second sheet 52. At the position of the front opening portion 52a on the front surface side of the bag-shaped second sheet 52, the separator 22 may be disposed between the inside and outside of the bag, and the operation from the medical robot 500 can be transmitted from the inside of the bag to the outside of the bag via the separator 22.

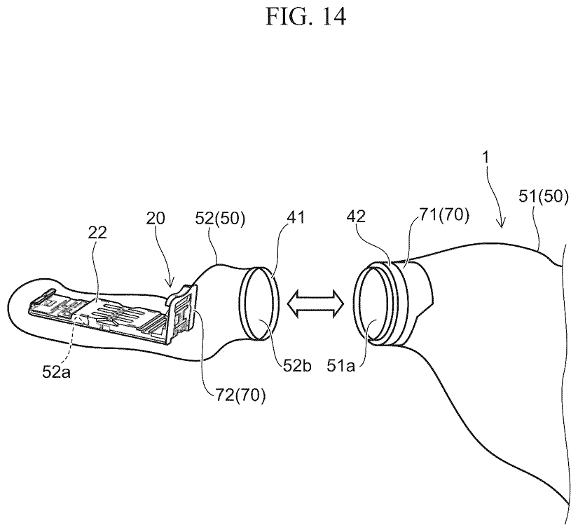

FIG. 14 is a schematic diagram of the coupling between the first sheet and the second sheet according to some embodiments.

The rotating member 41 attached to the rear opening portion 52b of the second sheet 52 is coupled with the joint member 42 of the first sheet 51. As described previously, the rotating member 41 is rotatably coupled with the joint member 42. This configuration allows the second sheet 52 side to be rotatable even though the first sheet 51 and the second sheet 52 are integrated. When the holder 21 portion provided at the tip of the arm 510 of the medical robot 500 rotates, the second sheet 52 also rotates together with the separator 22, and the occurrence of twisting of the drape 1 can be prevented.

### First Pinch Prevention Part

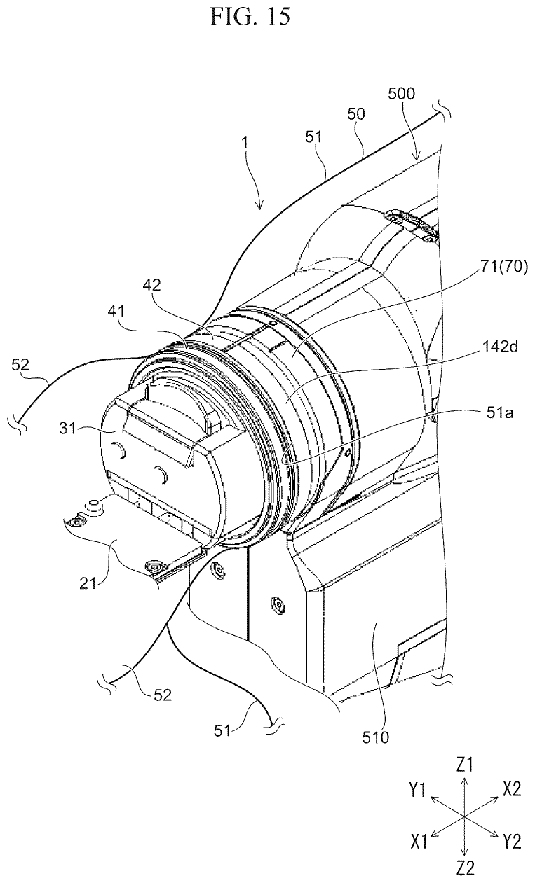

FIG. 15 is a perspective view of an attachment portion of the first sheet according to some embodiments.

The first opening portion 51a of the first sheet 51 may be fixed along the outer peripheral surface 142d of the joint member 42. The first pinch prevention part 71 may be a flexible body that is provided between the first sheet 51 and the outer peripheral surface 142d of the joint member 42.

In some embodiments, the first pinch prevention part 71 may include a tube that is provided along the outer peripheral surface 142d of the joint member 42, and may have an extended portion that extends rearward, i.e., toward the medical robot 500. With this configuration, when the first sheet 51 is placed over the holder 21 provided at the tip of the arm 510 of the medical robot 500, the first pinch prevention part 71 (e.g., the tube) can prevent jamming of the first sheet 51 on the outer periphery of the joint member 42.

That is, the first sheet 51 may be provided in a size that covers the arm 510, so it is provided in a large bag shape rearward from the joint member 42. The joint member 42 may be fitted to the base member 43 in a state in which the bag-shaped first sheet 51 is placed over the arm 510. In this operation, the first sheet 51 is likely to be jammed between the joint member 42 and the base member 43. In some embodiments, since the joint member 42 is provided with the first pinch prevention part 71 (e.g., the tube), the space between the joint member 42 and the base member 43 is covered with the first pinch prevention part 71 (e.g., the tube), and the first sheet 51 can be prevented from being pinched between the joint member 42 and the base member 43.

In an embodiment, the flexibility of the first pinch prevention part 71 (e.g., the tube) is higher than that of the joint member 42 and lower than that of the sheet 50 (first sheet 51). This configuration allows the extended portion of the first pinch prevention part 71 to serve as a support and prevent the first sheet 51 from approaching the joint member 42. Therefore, when the joint member 42 is fitted to the base member 43 in a state in which the sheet 50 is placed over the medical robot 500, the first sheet 51 can be prevented from approaching the edge of the joint member 42 and can also be prevented from being pinched between the joint member 42 and the base member 43.

In an embodiment, at least the extended portion of the first pinch prevention part 71 (e.g., the tube) may be translucent. Therefore, when the joint member 42 is fitted to the base member 43, it is easy to refer to the fitting situation through the first pinch prevention part 71 having translucency.

Thus, according to some embodiments, the clean area and the unclean area can be sufficiently isolated, and when the drape 1 is attached to the medical robot 500, the sheet 50 of the drape 1 can be avoided from being pinched, and it is possible to prevent poor fixation of the attachment 20 and damage to the drape 1.

While various embodiments have been described above with reference to the drawings, the present disclosure is not limited thereto, and any combination or substitution of components as appropriate is included within the scope of the present disclosure. In some embodiments, modifications such as combinations, changes in the order of processes, and various changes in design may be made on the basis of knowledge of a person skilled in the art, and such modified embodiments are within the scope of the present disclosure and the appended claims.

According to embodiments consistent with the present disclosure, the insertion member 100 may be a part of an endoscope.

What is claimed is:

1. A drape for covering a medical robot, the drape comprising:
an attachment detachably attached to the medical robot;
a flexible sheet covering the medical robot, the flexible sheet being fixed to the attachment; and
a flexible body that prevents the sheet from being pinched between the attachment and the medical robot.

2. The drape according to claim 1, wherein the flexible body has a flexibility greater than a flexibility of the attachment and less than a flexibility of the sheet.

3. The drape according to claim 1, wherein the flexible body is translucent.

4. The drape according to claim 1, wherein the flexible body comprises a plate-shaped body that has an extending portion that extends outward from an outer surface of the attachment.

5. The drape according to claim 4, wherein the plate-shaped body is fixed to the sheet in an area including an end portion of the extended portion.

6. The drape according to claim 1, wherein a first adhesive layer is provided on at least a part of an outer surface of the flexible body, the sheet is attached to the flexible body by the first adhesive layer.

7. The drape according to claim 6, wherein the flexible body comprises a plate-shaped body that has an extended portion that extends outward from an outer surface of the attachment, and the first adhesive layer is provided on a surface of the plate-shaped body and extends to an end portion of the extended portion of the plate-shaped body.

8. The drape according to claim 6, wherein the drape further comprises a second adhesive layer provided on a side of the sheet opposite to a side to which the first adhesive layer is attached, and the sheet comprises an interposed portion between the first adhesive layer and the second adhesive layer.

9. The drape according to claim 8, wherein the attachment is on a side of the second adhesive layer opposite to a side facing the sheet.

10. The drape according to claim 8, wherein a cover film is provided on a side of the second adhesive layer opposite to a side facing the sheet and configured to protect the second adhesive layer.

11. The drape according to claim 8, wherein the interposed portion of the sheet comprises a folded portion of the sheet.

12. The drape according to claim 1, wherein the drape is further configured to cover a holder at an end of an arm of the medical robot, the holder being detachable from the end of the arm, rotatable around the arm, and configured to hold an insertion member to be inserted into a patient,
wherein the attachment comprises a bearing structure, and the bearing structure comprises:
a ring-shaped fixation member that is attached to the arm in a state in which the holder is inserted into the fixation member; and
a ring-shaped rotating member that is slidably and rotatably attached to the fixation member in a state in which the holder is inserted into the rotating member and that is rotatable together with the holder, and
wherein the sheet comprises:
a first sheet that is attached to the fixation member and that is configured to cover the arm; and
a second sheet that is attached to the rotating member and rotatable together with the rotating member, and that is configured to cover the holder.

13. The drape according to claim 12, wherein the flexible body comprises a tube that is provided on an outer periphery of the fixation member.

14. The drape according to claim 13, wherein the tube extends in a direction away from the rotating member.

15. The drape according to claim 12, wherein the rotating member comprises a recessed portion on an outer periphery of the rotating member in which an adhesive layer is disposed, and
wherein the adhesive layer is configured to attach the rotating member to the second sheet.

16. The drape according to claim 1, wherein the attachment comprises a separator is disposed between the medical robot and an insertion member, the insertion member being held by the medical robot and configured to be inserted into a patient, the separator being configured to isolate the insertion member from the medical robot and transmit power in forward and backward directions from a power transmitter of the medical robot to a movable part of the insertion member, and
wherein the separator comprises:
a slider having a first engagement portion engaged with the power transmitter and a second engagement portion engaged with the movable part of the insertion member, the slider being configured to transmit the power received from the power transmitter to the movable part; and
a separator main body comprising a through-hole through which a part of the slider is inserted, the separator main body being detachably attached to the medical robot.

17. The drape according to claim 16, wherein: the drape comprises a first adhesive layer provided on a side of the flexible body facing the separator main body and a second adhesive layer provided on a side of the separator main body facing the flexible body, and
wherein the sheet comprises an interposed portion interposed between the first adhesive layer and the second adhesive layer.

18. The drape according to claim 17, wherein the flexible body comprises an opening portion, and wherein the sheet is attached to the first adhesive layer so as not to cover the opening portion.

19. The drape according to claim 16, wherein the flexible body comprises a plate-shaped body.

20. The drape according to claim 19, wherein an edge of a portion of the plate-shaped body located around the slider is coplanar with an edge the separator main body located around the slider.
